(19) United States
(12) Patent Application Publication
(10) Pub. No.: US 2011/0085140 A1
(43) Pub. Date: Apr. 14, 2011

(54) APPARATUS AND METHODS FOR TESTING CONTRAST SENSITIVITY

(75) Inventors: Angela M. Brown, Worthington, OH (US); Delwin T. Lindsey, Worthington, OH (US)

(73) Assignee: THE OHIO STATE UNIVERSITY RESEARCH FOUNDATION, Columbus, OH (US)

(21) Appl. No.: 12/901,009

(22) Filed: Oct. 8, 2010

Related U.S. Application Data
(60) Provisional application No. 61/250,076, filed on Oct. 9, 2009.

(51) Int. Cl. A61B 3/06

(52) U.S. Cl. 351/239; 351/246

(57) ABSTRACT

A test for measuring the contrast sensitivity of a patient uses a set of card that each include a stimulus, such as a square wave grating, presented at a single low-spatial frequency. The gratings vary in contrast from card to card. The test allows for the determination of the maximum contrast sensitivity of the patient in a single measurement, without knowing the spatial frequency at which that maximum occurs, which is possible because the spatial frequency is low enough that it is most likely below the maximum of the contrast sensitivity function in patients of any age.

### RELATED APPLICATION

The present application is being filed as a non-provisional patent application claiming priority/benefit under 35 U.S.C. §119(e) from the U.S. provisional patent application having Ser. No. 61/250,076 and filed on Oct. 9, 2009, the entire disclosure of which is herein incorporated by reference in its entirety.

### FIELD

The general inventive concepts relate to human vision testing and, more specifically, to improved apparatus and methods for measuring a person's contrast sensitivity.

### BACKGROUND

The contrast of a visual stimulus is the amount of modulation between light and dark across its area. The modulation may be in the form of a sine wave, a square wave, or a more complex pattern such as a letter. A high-contrast stimulus has bold areas of white and black, and a low-contrast stimulus has subtle variations of lighter and darker gray areas. In vision research, contrast is defined as the luminance increment, divided by the average luminance over the whole stimulus (equivalently, (max−min)/(max+min)) in the case of a sine-wave or square wave stimulus). An observer's contrast threshold is defined as the contrast of the lowest-contrast, just-visible stimulus, with the observer's contrast sensitivity being the reciprocal of that value.

The contrast sensitivity function shows contrast sensitivity as a function of the spatial frequency of the sinusoidally-modulated luminance grating that is used to measure it. It is a single-peaked function, with two key parameters: its overall level, which is captured by the sensitivity at its peak, and its lateral position, which is captured by the cutoff spatial frequency, or “grating acuity,” which is the highest spatial frequency that the observer can resolve when non-coherent light is used. Much of the variability among low-vision observers is captured by these two parameters.

A patient's contrast sensitivity can be impaired even when his/her visual acuity is nearly normal. Visual acuity predicts visual disability when the acuity loss is considerable, but individuals with only modest visual acuity loss may be very disabled if their contrast sensitivity is reduced.

There are many reasons for measuring contrast sensitivity. While visual acuity is the standard measure of visual performance for diagnostic and legal purposes, contrast sensitivity may be a better predictor of the depth of visual handicap experienced in daily life, especially in the case of patients with serious vision loss. For example, many tasks in the daily life of a low-vision individual are more limited by reduced contrast sensitivity than by reduced visual acuity, and the individual with reduced visual acuity can be handicapped disproportionately if his or her contrast sensitivity is also poor.

Additionally, measuring contrast sensitivity may be useful in diagnosing or otherwise predicting certain visual diseases and disorders. Furthermore, in recent years, new treatments for retinal diseases and disorders have been developed and others are under development, and there is a need for additional modalities such as contrast sensitivity for evaluating the visual performance of the patients receiving these treatments, before, during, and after treatment. Accordingly, effective evaluation of treatments for visual diseases and disorders could benefit from a convenient method of measuring contrast sensitivity.

As another example, contrast sensitivity can be a limiting factor in a person's ability to learn to read. Educators understand that the beginning reader needs big letters, but they may overlook the fact that contrast is important too. Letters written in pencil or chalk, or using narrow marks on a whiteboard, will all be low in contrast no matter how big they are, and will challenge the visual capabilities of the student with poor contrast sensitivity. Thus, contrast sensitivity testing of students with reduced vision can facilitate making appropriate accommodations to promote effective education of these students.

A Pelli-Robson contrast sensitivity chart has conventionally been used by clinicians to measure a subject's contrast sensitivity. A Pelli-Robson chart 100 is shown in FIG. 1. The Pelli-Robson test measures contrast sensitivity using a single large letter size (20/60 optotype), with contrast varying across groups of letters. Specifically, the chart 100 uses letters 102 (6 per line) arranged in groups 104 whose contrast varies from high to low. Patients read the letters 102, starting with the highest contrast, until they are unable to read two or three letters 102 in a single group 104. Each group 104 has three letters 102 of the same contrast level, so there are three trials per contrast level. The subject is assigned a score based on the contrast of the last group 104 in which two or three letters 102 were correctly read. For example, according to one scoring strategy, the subject is assigned a single number, as the score, which is a measure of the subject's log contrast sensitivity. Thus a score of 2.0 means that the subject was able to read at least two of the three letters with a contrast of 1 percent (contrast sensitivity=100). A Pelli-Robson score of 2.0 indicates normal contrast sensitivity of 100. Scores less than 2.0 signify poorer contrast sensitivity. A Pelli-Robson contrast sensitivity score of less than 1.5 is consistent with visual impairment and a score of less than 1.0 represents visual disability. A score of 1.0 represents an approximately 10-fold loss of contrast sensitivity. That is, a person with a contrast sensitivity of 1.0 requires 10 times as much contrast to see as compared with a person with normal vision. A loss of this magnitude would be quite disabling and would have a huge impact on one's ability to drive or read.

Another conventional tool for measuring the contrast sensitivity of a subject is the Vision Contrast Test System (VCTS®) provided by Vistech Consultants, Inc. The Vistech system utilizes a chart. A Vistech chart 200 is shown in FIG. 2. The Vistech chart 200 is made up of a number of rows 202 (e.g., 5 rows) of sine wave gratings 204, with each of the sine wave gratings 204 having the same diameter (e.g., a 3-inch diameter). In each row 202, the gratings 204 are provided at a given spatial frequency but differ in contrast. Different spatial frequencies are utilized between the rows 202. For example, from a top row (A) to a bottom row (E), the gratings 204 can have a spatial frequency of 1, 2, 4, 8, and 16 cycles per degree, respectively. Each grating 204 is oriented in one of 3 directions: vertical, slanted 15 degrees to the left, or slanted 15 degrees to the right. The task of the patient is to report the orientation of each grating 204 in each row 202 until the orientation cannot be determined. When the test is completed, the data are plotted and compared to a “normal” contrast sensitivity curve. The Vistech charts include: the VCTS®-6500 for distance testing and the VCTS®-6000 for near testing.

Another conventional tool for measuring the contrast sensitivity of a subject is the Cambridge contrast chart. A Cambridge contrast chart in the form of a spiral-bound book 300 is shown in FIG. 3. In this test, the spiral-bound book 300 is A4 in size (28 cm×22 cm) and includes several pages 302 bound by a spiral connector 304. The pages 302 include square wave gratings 306 of decreasing contrast that are used to measure a single median spatial frequency (4 cycles per degree at six meters). Although the Cambridge gratings 306 measure contrast sensitivity at only one spatial frequency (i.e., 4 cycles per degree), when sensitivity to other spatial frequencies is impaired sensitivity at 4 cycles per degree is usually also affected. The gratings are composed of fine dots 308, which while not visible at six meters, may be visible at shorter distances. The lightness of the square-wave stripes is manipulated by the density of the dots 308, which depends on the patient being far enough away so that the dots 308 are not visible. Consequently, the square wave is generally at or above the peak of the contrast sensitivity function.

Each grating 306 is presented on one page 302 with a facing page (not shown) of uniform grey and the patient must indicate on which side the grating is seen. This is a true forced choice procedure. Gratings 306 are presented until an incorrect response is made, this is then repeated, and the sum of four incorrect responses is recorded. The normal score (total of four responses) should reduce from about 35 at age 25 years to about 29 at age 70 years. Similarly, an abnormal score (95 per cent confidence limit) is reported to reduce from a score of less than 27 at age 25 years to less than 23 at age 70 years. The luminance requirement of 100 cd/m² is relatively easily met on this small chart.

Another conventional tool for measuring the contrast sensitivity of a subject is the Melbourne Edge Test (MET). This test uses a single stimulus (i.e., an edge between light and dark semicircles) to measure global contrast sensitivity. The current version of the MET uses a relatively small, portable electronic device to present the stimuli. A light box 400 having a screen 402 for use in the MET is shown in FIG. 4. Additionally, a stylus 404 or similar pointing device can be used to interact with the screen 402. The screen 402 can be backlit by a light source (not shown) within the light box 400. The screen 402 includes 15 disks 406 displayed thereon (i.e., three lines of five disks each). Each disk contains a stimuli (i.e., an edge between semicircles of differing contrasts) of varying orientation (i.e., positive slope, vertical, negative slope, or horizontal), wherein the stimuli of each subsequent disk 406 has a lower contrast differential than its predecessor. Like with the Vistech chart, the patient views the stimuli of the disks 406 and identifies the orientation of each.

Another conventional tool for measuring the contrast sensitivity of a subject is the card-based system developed by Drs. Russell Adams and Mary Courage (hereinafter the Adams card test). The Adams card test consists of 40 large (56 cm×28 cm) matteboard cards. An Adams card 500 is shown in FIG. 5. The card 500 contains two circular gratings 502, 504 located to the left and right of a central peephole 506, respectively. The gratings 502, 504 subtend a visual angle of 16.3° from the testing distance of 60 cm. One grating, the test grating 502, is a sine wave grating of a given spatial frequency and contrast. The other grating, the control grating 504, is a sine wave grating of the same spatial frequency, but with a contrast of 0% (i.e., all stripes are of equal luminance) and thus is indiscriminable from the background 508 of the card 500. A zero-contrast stimulus is used as a control grating to ensure that observers do not detect the position of the test grating by relying on edge/grating artifacts. Under testing conditions, the space average luminance of each grating 502, 504 and the background 508 of each card 500 was 22.0 cd/m². The cards 500 are divided into five sets of eight based on the spatial frequency of the gratings 502, 504 in each set (0.4, 0.8, 1.6, 3.2, or 6.4 cycles per degree). Within each set, contrast levels of the test gratings 502 vary from a maximum of 55% (the warm-up card) to a minimum of 1.4%. The average contrast step size between adjacent cards 500 in each set is 0.16 log contrast sensitivity units (range, 0.14-0.21 log contrast sensitivity units). The cards 500 are presented within an opening in a three-panel matteboard backboard that approximates the average luminance of the cards. In measuring the contrast sensitivity of a patient using the Adams card test, the highest contrast card from one of the spatial frequency sets can be initially presented repeatedly and rotated until the tester can decide whether the patient shows a consistent preference for one side of the card. This procedure continues with the remaining cards in the set until the patient demonstrates no preference for either side of the card. The lowest contrast grating detected by the patient is taken as an estimate of contrast threshold for that spatial frequency.

Other conventional tests for measuring the contrast sensitivity of a subject include the Cardiff Contrast Sensitivity Test, the Hiding Heidi test, the Mr. Happy test, the Lea Symbols test, and the Mars Letter Contrast Sensitivity Test.

The conventional approaches to testing and measuring contrast sensitivity all suffer from drawbacks that, for example, limit their effectiveness, accuracy, and/or applicability.

For example, in the Pelli-Robson contrast sensitivity test, the patient reads the letters on the chart until the contrast becomes so low that he/she makes errors. This test is a three-trial, 26-alternative forced-choice task (i.e., the patient chooses among the 26 known letters, although fewer letters actually occur in the chart). Several scoring protocols are in use for using the resulting performance to arrive at a measure of contrast sensitivity. The Pelli-Robson chart, however, cannot be used on infants or any patient who cannot read letters.

In the Vistech VCTS, the patient examines each of the rows of the disks, with each disk containing a grating in one of three orientations (positive slope, vertical, or negative slope). Each row portrays a different spatial frequency, and each grating within the row has a lower contrast than its predecessor. The patient must identify the orientation of each grating. Thus, the Vistech chart uses a single 3-alternative forced-choice judgment at each contrast level and spatial frequency to measure the patient's contrast sensitivity. The Vistech VCTS, however, is drastically under-powered for many statistical purposes. Furthermore, because the Vistech VCTS requires that the patient identify the orientation of each element in a row, over several rows of elements, the test is not suitable for infants or other uninstructable patients.

In the Cambridge gratings test, the gratings are designed to be at a spatial frequency near the maximum of the contrast sensitivity function. However, in a clincal population, the spatial frequency of the maximum of the contrast sensitivity function cannot be known in advance. If the spatial frequency is misjudged, and (as will often be the case) the gratings are too high in spatial frequency, the contrast sensitivity of the patient will be underestimated. Furthermore, because the stimuli (i.e., the gratings) are provided in a spiral-bound notebook, young patients (e.g., infants) may look at the spiral rather than the stimulus.

In the Melbourne Edge Test, a single stimulus is used to measure global contrast sensitivity. Like with the Vistech chart, the patient views a series of disks containing stimuli of different orientations (positive slope, vertical, negative slope, or horizontal), and he/she identifies the orientation of each. Because the Melbourne Edge Test requires that the patient indentify the orientation of each element in a row, it is not suitable for testing infants or other uninstructable patients.

In the Adams card test, gratings of variable contrast are placed on cards. The gratings span several spatial frequencies that are sinusoidally modulated in luminance. Thus, the Adams card test requires tests at multiple spatial frequencies to determine the spatial frequency at which maximum contrast sensitivity occurs and to determine that contrast sensitivity value. As a result, the test either takes a long time (e.g., 4 or more measurements are typically required to find the maximum) or, in the interests of speed, the measurements may be rushed thereby sacrificing the accuracy of the assessment. Additionally, multiple determinations of the maximum contrast sensitivity may need to be acquired (and averaged) to obtain a reliable measurement, which further increases the time it takes to complete the test. Because the Adams card test requires many measurements distributed over multiple testing sessions (e.g., multiple appointments), they are typically cost prohibitive, as well as an added inconvenience for the patient.

There is a need for apparatus and methods for measuring the contrast sensitivity of individuals, which improve upon one or more of the above identified and/or other limitations of conventional contrast sensitivity tests.

### SUMMARY

The general inventive concepts encompass apparatus and methods for measuring, determining, or otherwise estimating the contrast sensitivity of an individual.

The general inventive concepts encompass apparatus and methods for measuring the contrast sensitivity of an individual using a single, low-spatial frequency stimuli. In one exemplary embodiment, the low-spatial frequency stimuli are square wave gratings.

The general inventive concepts encompass apparatus and methods for determining the contrast sensitivity of an individual using a single measurement.

The general inventive concepts encompass apparatus and methods for determining the contrast sensitivity of an individual as a measure of the individual's overall visual ability. In one exemplary embodiment, a measure of an individual's contrast sensitivity is analyzed in conjunction with a measure of the individual's visual acuity to determine or otherwise approximate the individual's overall visual ability.

The general inventive concepts encompass apparatus and methods for measuring the contrast sensitivity of an individual for diagnosing and/or predicting a visual disease or disorder.

The general inventive concepts encompass apparatus and methods for measuring the contrast sensitivity of an individual for evaluating the effectiveness of a treatment for a visual disease or disorder. In one exemplary embodiment, the contrast sensitivity of the individual is evaluated before, during, and after the treatment.

In one exemplary embodiment, the visual disease or disorder is a disease or disorder that particularly afflicts infants and children, such as Retinopathy of Prematurity, Cortical Vision Impairment, retinal degenerations (e.g., Leber's Congenital Amaurosis and Bardet-Biedl Syndrome), Congenital Cataract, and Amblyopia.

The general inventive concepts encompass apparatus and methods suitable for measuring the contrast sensitivity of a handicapped, illiterate, uninstructable (i.e., incapable of being instructed), or nonverbal individual. In one exemplary embodiment, the individual is a child 3 years old or younger. In one exemplary embodiment, the individual is a child 1 year old or younger.

The general inventive concepts encompass apparatus and methods suitable for the monocular and binocular testing of the contrast sensitivity of an individual.

In one exemplary embodiment of the general inventive concepts, a method for measuring a contrast sensitivity of a patient is disclosed. The method comprises: showing the patient a plurality of cards, each card including a square wave grating having a spatial frequency and a contrast; observing a behavioral response of the patient to each of the cards; and evaluating the contrast sensitivity of the patient based on the behavioral responses, wherein the spatial frequency of each square wave grating is the same, and wherein the contrast of each square wave grating is different. The method is useful for measuring the contrast sensitivity of a patient of any age, including children 3 years old and younger; patients 3 years old or older if handicapped, illiterate, or uninstructable; and non-verbal patients of any age.

In one exemplary embodiment, the low spatial frequency is one that allows three full cycles (relative to a size of the stimulus and a predefined or expected testing distance) to appear on a card that has the desired dimensions suitable for the effective testing of patients. In one exemplary embodiment, the spatial frequency is less than 0.250 cycles per degree (relative to a size of the stimulus and a predefined or expected testing distance). In one exemplary embodiment, the spatial frequency is approximately 0.125 cycles per degree (relative to a size of the stimulus and a predefined or expected testing distance).

In one exemplary embodiment, a size of each card is approximately 25.5 cm×56.0 cm. In one exemplary embodiment, each square wave grating occupies approximately half of its corresponding card. In one exemplary embodiment, each square wave grating is tapered towards a center of its corresponding card. In one exemplary embodiment, each card includes an aperture in its middle for observing the behavioral response of the patient to the card.

In one exemplary embodiment of the general inventive concepts, a system for measuring a contrast sensitivity of a patient is disclosed. The system comprises: a plurality of cards, each card including a square wave grating having a spatial frequency and a contrast, wherein the spatial frequency of each square wave grating is the same, and wherein the contrast of each square wave grating is different.

In one exemplary embodiment, the spatial frequency is less than 0.125 cycles per degree. In one exemplary embodiment, the spatial frequency is approximately 0.125 cycles per degree.

In one exemplary embodiment, a size of each card is approximately 25.5 cm×56.0 cm. In one exemplary embodiment, for each of the cards: the square wave grating occupies approximately one half of the card and a uniform gray color with a reflectance of 50% occupies approximately one half of the card.

In one exemplary embodiment, each square wave grating is tapered towards a center of its corresponding card. In one exemplary embodiment, each card includes an aperture that is operable to allow a person to see through the card.

In one exemplary embodiment of the general inventive concepts, an apparatus for measuring a contrast sensitivity of a patient is disclosed. The apparatus comprises: a substrate, the substrate including a low spatial frequency square-wave grating with a predetermined contrast. The substrate can be paper, plastic, or any other rigid, lightweight material suitable for having the grating displayed thereon. In one exemplary embodiment, the substrate measures approximately 25.5 cm×56.0 cm.

In one exemplary embodiment, the low spatial frequency is one that allows three full cycles (relative to a size of the stimulus and a predefined or expected testing distance) to appear on a card that has the desired dimensions suitable for the effective testing of patients. In one exemplary embodiment, the spatial frequency is less than 0.250 cycles per degree (relative to a size of the stimulus and a predefined or expected testing distance). In one exemplary embodiment, the spatial frequency is approximately 0.125 cycles per degree (relative to a size of the stimulus and a predefined or expected testing distance).

In one exemplary embodiment, the square wave grating is tapered towards a center of the substrate. In one exemplary embodiment, the substrate includes an aperture that is operable to allow a person to see through the substrate.

Numerous other aspects, advantages and/or features of the general inventive concepts will become more readily apparent from the following detailed description of exemplary embodiments, from the claims, and from the accompanying drawings and related papers being submitted herewith.

### BRIEF DESCRIPTION OF THE DRAWINGS

The general inventive concepts as well as embodiments and advantages thereof are described below in greater detail, by way of example, with reference to the drawings in which:

FIG. 1 is an image of a conventional Pelli-Robson chart used in measuring contrast sensitivity.

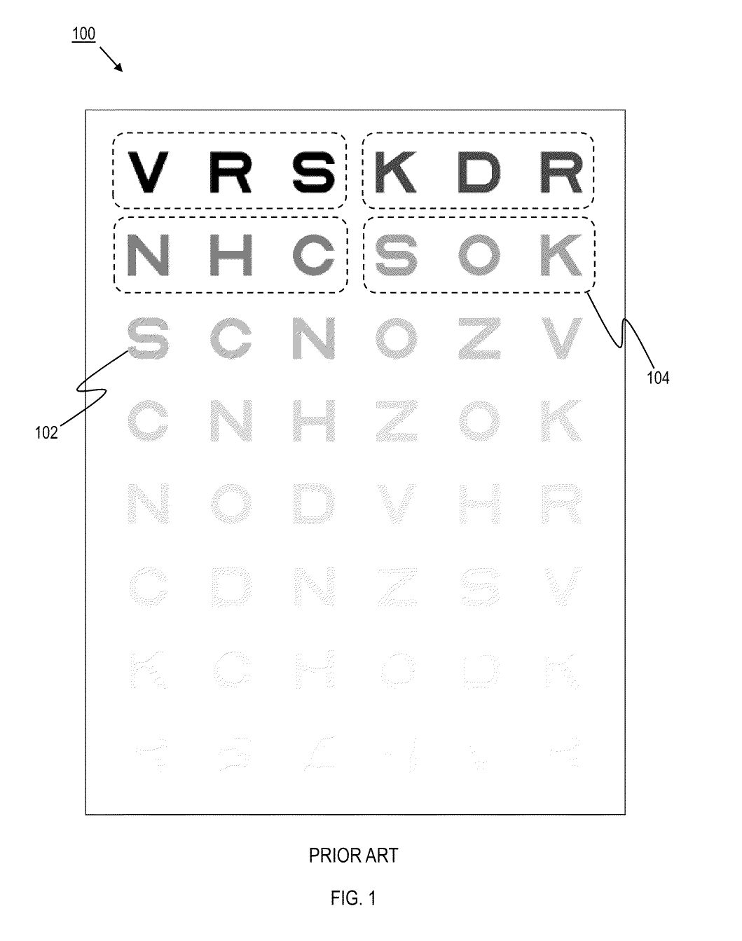

FIG. 2 is an image of a conventional Vistech chart used in measuring contrast sensitivity.

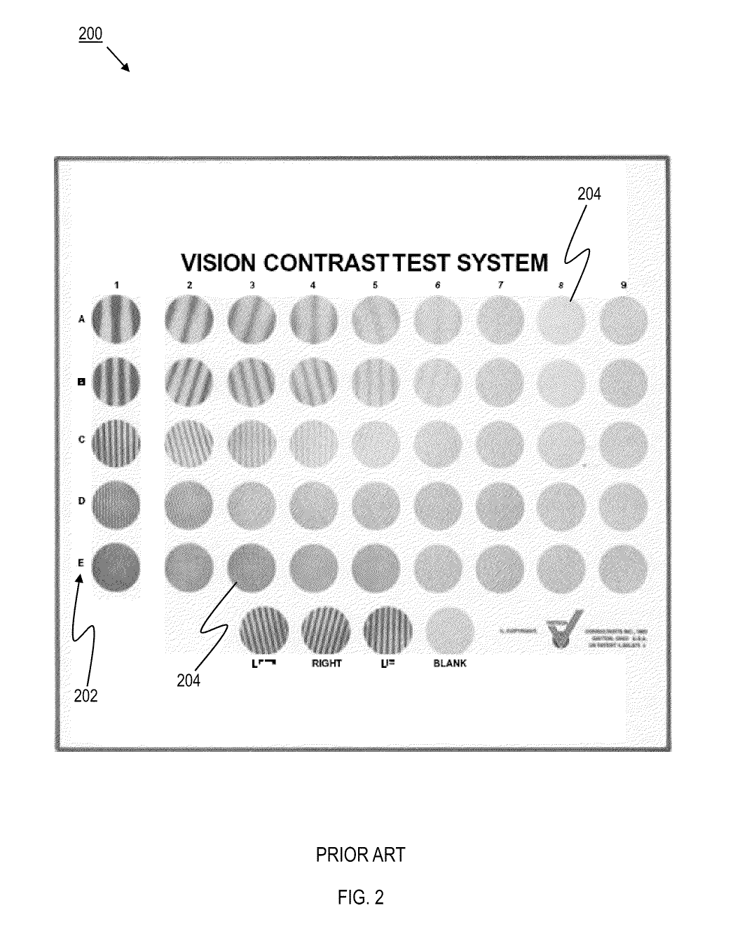

FIG. 3 is an image of a conventional Cambridge contrast book, in both a closed and an opened configuration, and superimposed on dots making up the lines, used in measuring contrast sensitivity.

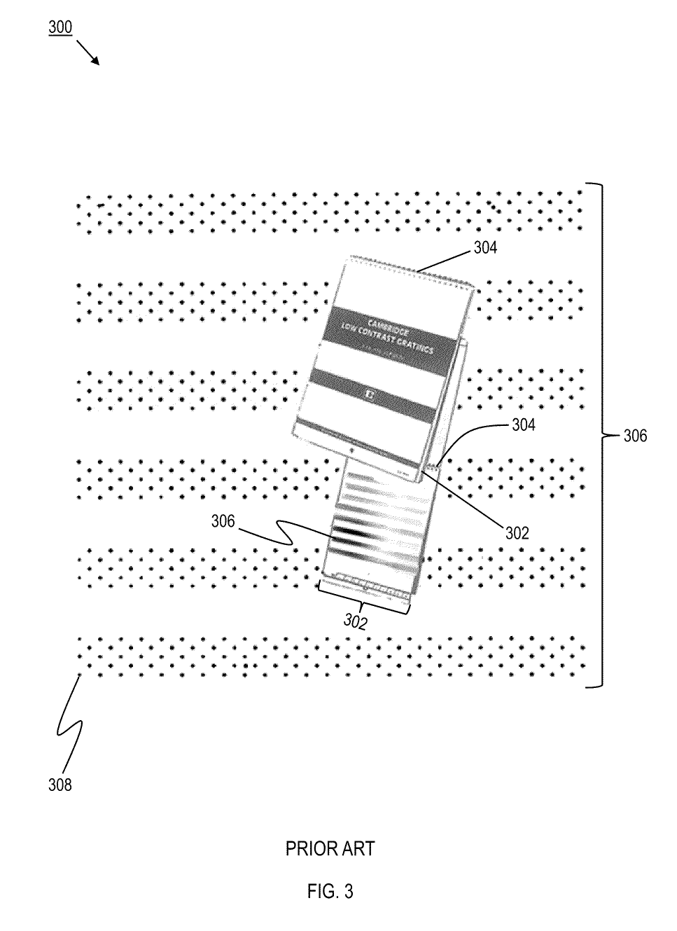

FIG. 4 is an image of a conventional Melbourne Edge Test light box used in measuring contrast sensitivity.

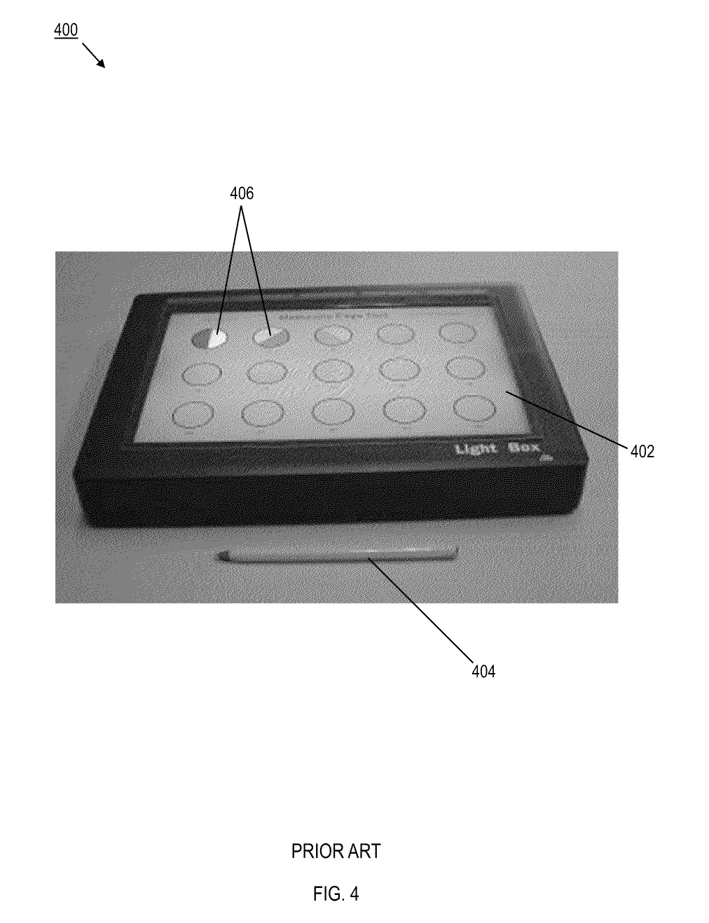

FIG. 5 is an image of a conventional Adams card used in measuring contrast sensitivity.

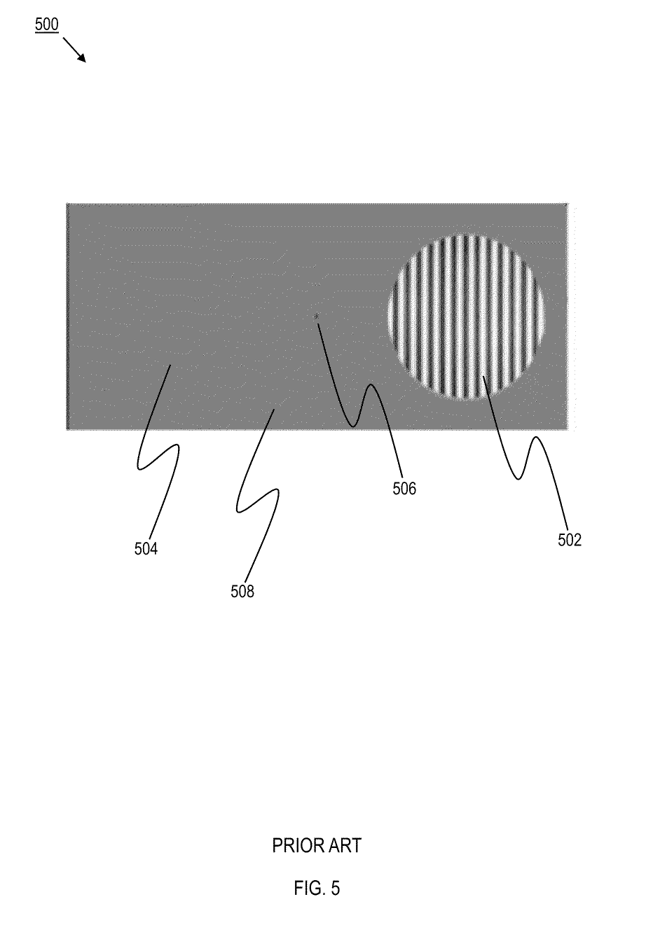

FIG. 6 is an image of striped cards, according to one exemplary embodiment, for use in measuring contrast sensitivity of a patient.

FIG. 7 is a graph illustrating derivation of the contrast sensitivity function (CSF) of a patient to a square wave grating, according to one exemplary embodiment.

FIG. 8 is a flowchart illustrating a method of measuring the contrast sensitivity of a patient, according to one exemplary embodiment.

### DETAILED DESCRIPTION

While the general inventive concepts are susceptible of embodiment in many different forms, there are shown in the drawings and will be described herein in detail specific embodiments thereof with the understanding that the present disclosure is to be considered as an exemplification of the principles of the general inventive concepts. Accordingly, the general inventive concepts are not intended to be limited to the specific embodiments illustrated herein.

Unless suggested otherwise by the context in which they are used, the terms individual, subject, patient, observer, and the like are all intended to refer generally herein to a person having, or capable of having, their visual contrast sensitivity tested. Unless suggested otherwise by the context in which they are used, the terms tester, examiner, administrator, and the like all intended to refer generally herein to a person administering, or qualified to administer, the disclosed contrast sensitivity tests.

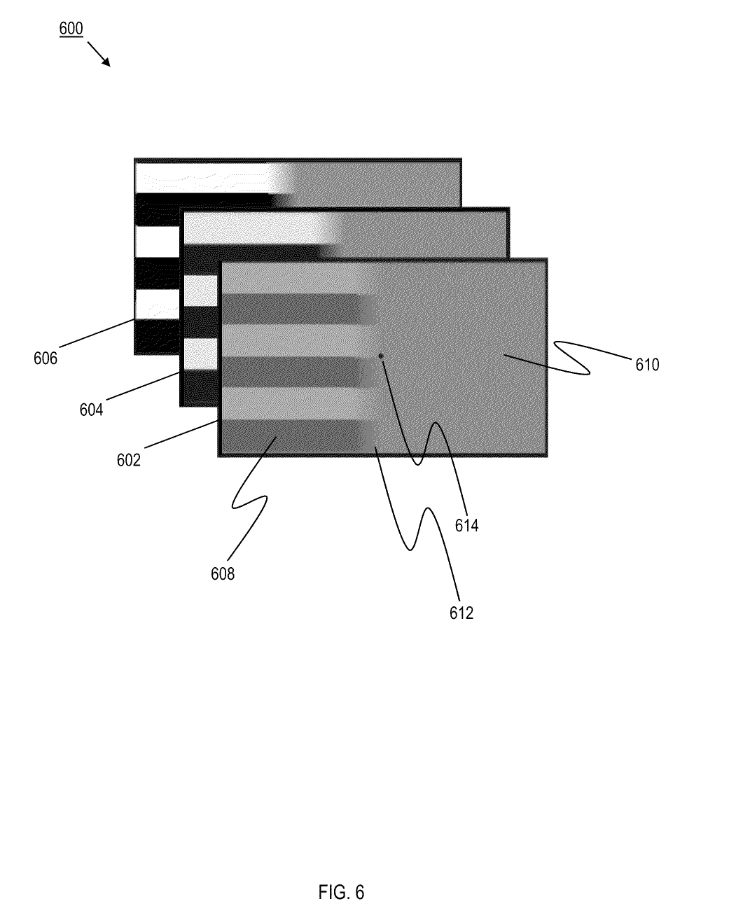

A contrast sensitivity test 600 (hereinafter the Stripe Card Contrast Sensitivity test or SCCS test) utilizes a set of cards. Three cards 602, 604, and 606 from the set of cards, according to one exemplary embodiment, are shown in FIG. 6. Each of the cards (e.g., cards 602, 604, and 606) is relatively large, yet sized so as to be readily held and manipulated by a person administering the test 600. In one exemplary embodiment, each of the cards is rectangular and measures 25.5 cm×56.0 cm. Each of the cards (e.g., cards 602, 604, and 606) includes a low spatial frequency stimulus 608.

In one exemplary embodiment, the low spatial frequency is one that allows three full cycles (relative to a size of the stimulus and a predefined or expected testing distance) to appear on a card that has the desired dimensions suitable for the effective testing of patients. In one exemplary embodiment, the spatial frequency is less than 0.250 cycles per degree (relative to a size of the stimulus and a predefined or expected testing distance). In one exemplary embodiment, the spatial frequency is approximately 0.125 cycles per degree (relative to a size of the stimulus and a predefined or expected testing distance). In one exemplary embodiment, the stimulus occupies approximately half of the card.

The contrast of the low spatial frequency stimulus 608 varies from card to card in a graded series, with each stimulus 608 being paired with a constant uniform gray region 610 on the card having a reflectance equal to the average reflectance of the stimulus 608. The contrast of the stimulus 608 is tapered 612 towards a center of the card to minimize edge artifacts. The cards can be rotated so the stimuli is on the left or on the right. A response of the patient to the stimulus 608 is viewed through the peephole 614 in the center of the card by the person administering the test 600.

In the SCCS test 600, the patient's contrast sensitivity is measured using a dichotomous preferential looking method that uses only the innate orienting behavior of a human infant or the looking or pointing behavior of a child or adult patient. Thus, the SCCS test 600 combines an innovative stimulus (e.g., the low spatial frequency luminance square wave grating) with a method of behavioral testing to produce a test of contrast sensitivity that is relatively fast and easy to use, inexpensive to produce, and can be readily used to measure contrast sensitivity in patients who, because of age or handicap, cannot read an eye chart or similar device.

By measuring a patient's contrast sensitivity using a low spatial square wave target, the measured contrast sensitivity will be near the patient's peak sensitivity as measured by the sine wave contrast sensitivity function. Thus, the low spatial frequency square wave target will allow a clinician to measure the peak contrast sensitivity of an observer (e.g., an infant) without knowing the spatial frequency at which it occurs by varying only the contrast of a single, low-spatial-frequency square-wave target. The aforementioned combination of the low spatial frequency square wave target stimulus and the card testing procedure allows the contrast sensitivity of a patient to be measured in a few minutes. It also allows for the practical measurement of the contrast sensitivity of patients (e.g., infants, young children, disabled adults) in a clinical setting.

Thus, the general inventive concepts contemplate use of a square wave grating of low spatial frequency (i.e., having very broad stripes) to measure contrast sensitivity. As noted above, the contrast sensitivity of a patient, when it is measured using the square wave target, will be approximately equal to the value at the maximum of the patient's contrast sensitivity function, as long as the square wave spatial frequency is below the sine wave frequency at which that maximum occurs. In this manner, a single, low spatial frequency target can be used to measure (i.e., approximate) the peak contrast sensitivity of any patient without knowing at what spatial frequency that maximum occurs. This allows contrast sensitivity to be measured with one measurement (i.e., varying contrast at a constant, low spatial frequency), thereby making contrast sensitivity testing more practical for use in a clinical setting and more suitable for a wider range of patients.

Using a very low spatial frequency, sharp-edged square-wave stimulus, instead of letters or a sinusoidal stimulus of varying spatial frequency, can be advantageous given the mathematical nature of square waves. Like the Sloan letters and other optotypes, the spectrum of a square wave declines linearly with spatial frequency, although in the case of the square wave, the amplitude spectrum is discrete rather than continuous. Its spectrum can be calculated as follows. Suppose that the nominal contrast of the square wave ([max−min]/[max+min]) is C. Then the spectrum of the square wave will be (C*4/π) at frequency=F1 (i.e., its nominal spatial frequency and, by definition, its first harmonic), (C*4/π)/3 at F3=3F1, (C*4/π)/5 at F5=5F1, and so forth.

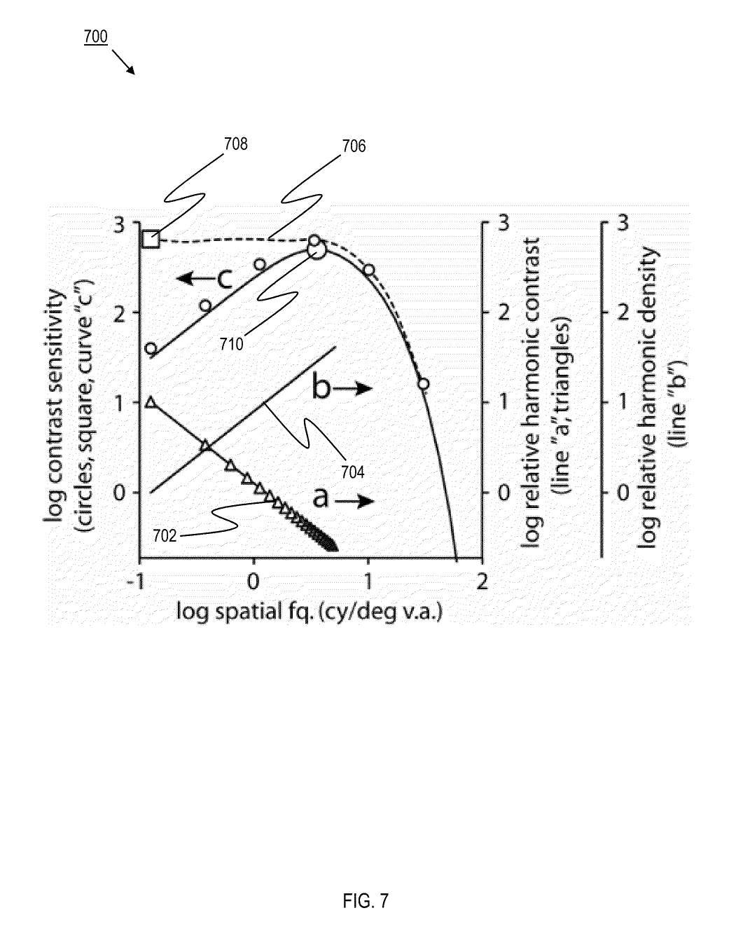

The derivation of an observer's sensitivity to a given stimulus proceeds in three steps, which will be described with reference to the graph 700 of FIG. 7.

First, the responses of the spatial frequency channels are estimated. Those responses will be the product of the contrast of the harmonics within the pass-bands of the channels, times the number of harmonics within their pass-bands, times the sensitivity of each pass-band. The contrast of the harmonics falls off linearly with spatial frequency, with a multiplicative constant of 4/π (as shown by line “a” 702 and the triangles in FIG. 7), and the density of the harmonics per pass-band rises linearly with spatial frequency (as shown by line “b” 704 in FIG. 7; notice also that the spacing of the triangles increases as spatial frequency increases), so the higher the spatial frequency a channel is tuned to, the lower the contrast of its harmonic content, but the more harmonics it contains. Thus, the total harmonic contrast within the pass-bands is approximately constant.

The second step is to choose the spatial frequency tuned channels. In one exemplary embodiment, the channels are chosen to have a full bandwidth of 1.6 octaves (0.482 common log units, or a linear factor if 3.03), with a peak-to-peak separation of one bandwidth. The responses of the channels to a just-detectable square-wave at 0.125 cycles per degree are shown by the small circles in FIG. 7.

In the third step, the sensitivity of the visual system to the square wave is calculated by combining the responses of the channels, for example, using a Quick pooling equation, i.e., Equation 1:

R_{full}=(ΣR_{channel}^{m})^{1/m} (1)

In this equation, the value of m is chosen to be approximately 4.

The predicted contrast sensitivity function for square-wave gratings is shown by the dashed line 706 in FIG. 7. The sensitivity of the eye to a square wave on the high spatial frequency limb of the contrast sensitivity function is higher than the sensitivity of the eye to a sine wave by a factor of about 4/π. In FIG. 7, the predicted sensitivity measured with a square wave of 0.125 cycles per degree (as shown by the large square 708) is near the peak sensitivity of the contrast sensitivity function as measured using sine waves (as shown by the large circle 710).

In view of the above, it is noted that contrast sensitivity measured using a low spatial-frequency square wave target is nearly constant with spatial frequency. Furthermore, this constant sensitivity value is near the maximum sensitivity of the sine-wave contrast sensitivity function. Thus, if the stimulus is a low-frequency square wave, the patient's peak contrast sensitivity can be measured without knowing the spatial frequency at which it occurs, as long as the spatial frequency of the square wave is low enough. In one exemplary embodiment, a square wave at 0.125 cycles per degree (relative to a size of the square wave and a predefined or expected testing distance) is chosen, because it is typically below the contrast sensitivity peak of the youngest patients (i.e., infants), and because that frequency allows three full cycles to appear on a card (e.g., card 602, 604, or 606) that has the desired dimensions suitable for the effective testing of patients.

In one exemplary embodiment, the low spatial frequency is one that allows three full cycles (relative to a size of the stimulus and a predefined or expected testing distance) to appear on a card that has the desired dimensions suitable for the effective testing of patients. In one exemplary embodiment, the spatial frequency is less than 0.250 cycles per degree (relative to a size of the stimulus and a predefined or expected testing distance). In one exemplary embodiment, the spatial frequency is approximately 0.125 cycles per degree (relative to a size of the stimulus and a predefined or expected testing distance).

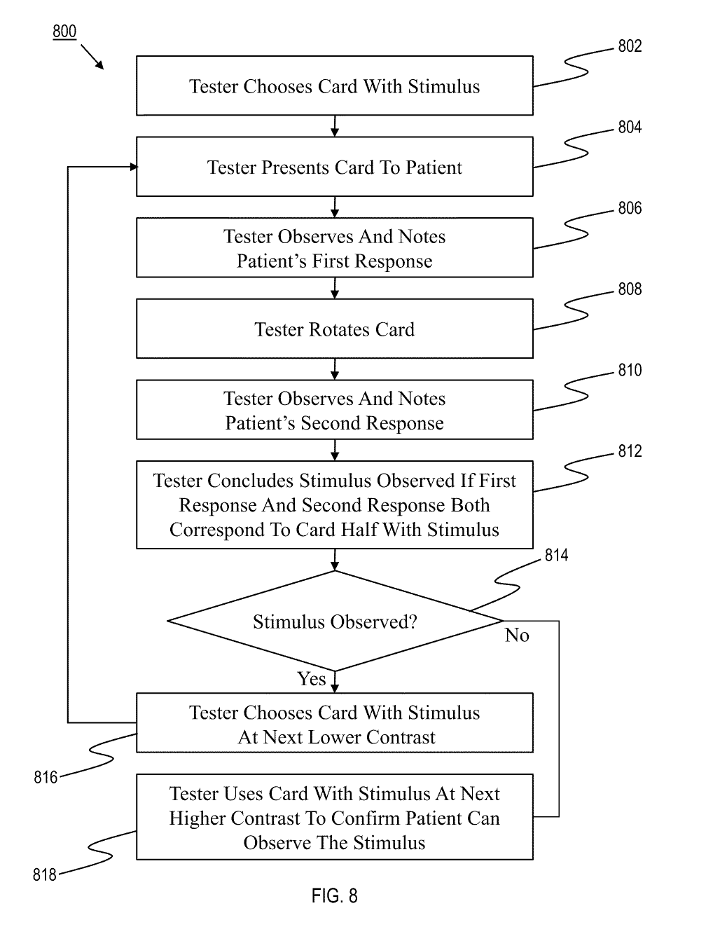

A method 800 (i.e., the SCCS test) for measuring the contrast sensitivity of a patient (e.g., an infant), according to one exemplary embodiment, will be described with reference to FIG. 8.

The method 800 utilizes a set of 21 cards (e.g., similar to the cards 602, 604, and 606). Each of the cards includes a very low spatial frequency square wave to measure contrast sensitivity of the patient. In one exemplary embodiment, the set of 21 cards includes a blank card for use in verifying a null response by the patient. The cards all have the same dimensions (e.g., 25.5 cm×56.0 cm). In one exemplary embodiment, the cards have the same dimensions as conventional Teller Acuity Cards, such that they could be used in conjunction with the Teller Acuity Cards and its associated puppet stage. One half of each card is uniform gray with a reflectance of near 50%, and the other half contains a square-wave grating of a calibrated contrast having a low spatial frequency equal to or less than 0.250 cycles per degree (see FIG. 6). The grating is tapered in contrast towards the midline so that the harmonics contained in the grating will almost all be from the square wave itself, rather than from artifacts at the ends of the stripes near the center of the screen. A small peephole is located in the center of the card to allow the person testing the patient to observe the patient's looking or pointing behavior without distracting the patient from the task.

The set of cards form a graded set spanning a card with the highest contrast (e.g., calibrated at 0.92 contrast) to a card with the lowest contrast (e.g., calibrated at 0.0076 contrast) in nominal steps. In one exemplary embodiment, a 0.1 log unit (e.g., 25%) step size is chosen because it spans the range with a reasonable number of cards (e.g., 20 cards), and because it is smaller than the standard deviation of the adult contrast sensitivity measured using the Pelli-Robson chart. If the grating appears visible (empirically) on the lowest contrast card, a card with a lower contrast grating can be made (e.g., by dithering (randomly half-toning) within 4×4 super-pixels) and included in the set.

The SCCS test facilitates testing of contrast sensitivity, using a single set of cards, over the entire range of ages from one-month-old infants up to normal adults. In the SCCS test, the person testing the patient (i.e., the tester) chooses a card, in step 802, whose grating should be easily visible to the patient. The tester presents the chosen card, in step 804, to the patient without looking at the grating or knowing which half it is on. In one exemplary embodiment, the card is presented to the patient within a window of a puppet stage or a similar structure for obscuring the tester.

In step 806, the tester observes the patient's looking or pointing behavior and makes a preliminary determination of which half of the card the patient prefers. Then the tester rotates the card, in step 810, so that the grating is on the opposite half (e.g., the left instead of the right, or vice versa) and presents it to the patient. In step 810, the tester again observes the patient's looking or pointing behavior and makes another determination of which half of the card the patient prefers. If the patient looks at or otherwise indicates a preference for the half of the card different from his or her previous preference (i.e., during the card's initial presentation), the tester concludes that the patient saw the grating, a conclusion that is checked by looking at the card and verifying that the grating was on the half of the card that the patient preferred both times, in step 812. If the tester confirms that the patient preferred the half of the card containing the stimulus in both orientations of the card (“YES” in step 814), the tester then chooses another (e.g., the next) lower contrast card, in step 816, and tests it using the same steps (i.e., steps 804, 806, 808, 810, and 812). Eventually, the tester will find a card including a stimulus that the patient cannot see (“NO” in step 814), as evidenced by a lack of preference for either half of the card under either orientation of the card. The tester then verifies, in step 818, that the patient can see the next highest contrast card. At the end of testing, the contrast sensitivity will be the contrast of the lowest contrast grating that the patient can see. Thus, the SCCS test can quickly and effectively measure the lowest contrast a patient can see by use of the cards having the low spatial frequency square-wave contrast stripes of variable contrast.

The method 800 is highly flexible and can be adapted by the tester as needed. For example, in step 804, the tester can elect to present the same card more than once, such as to confirm an initial observation by the tester. Again, if the tester doubts an initial observation, the tester can elect to “randomize” by placing it behind his or her back and rotating it several times until its orientation is unknown to the tester. Thereafter, the randomized card is presented to the patient again in step 804 and processing of the method continues therefrom. It will be appreciated that other steps of the method 800 can be adjusted without departing from the spirit and scope of the general inventive concepts.

The above description of specific embodiments has been given by way of example. From the disclosure given, those skilled in the art will not only understand the general inventive concepts and their attendant advantages, but will also find apparent various changes and modifications to the systems and methods disclosed. It is sought, therefore, to cover all such changes and modifications as fall within the spirit and scope of the general inventive concepts, as described and claimed herein, and any equivalents thereof

1. A method for measuring a contrast sensitivity of a patient, the method comprising:
showing the patient a plurality of cards, each card including a square wave grating having a spatial frequency and a contrast,
observing a behavioral response of the patient to each of the cards, and
evaluating the contrast sensitivity of the patient based on the behavioral responses,
wherein the spatial frequency of each square wave grating is the same, and
wherein the contrast of each square wave grating is different.

2. The method of claim 1, wherein the patient is 3 years old or younger.

3. The method of claim 1, wherein the patient is uninstructable.

4. The method of claim 1, wherein the patient is nonverbal.

5. The method of claim 1, wherein the spatial frequency is less than or equal to 0.250 cycles per degree.

6. The method of claim 1, wherein a size of each card is approximately 25.5 cm×56.0 cm.

7. The method of claim 1, wherein each square wave grating occupies approximately half of its corresponding card.

8. The method claim 1, wherein each square wave grating is tapered towards a center of its corresponding card.

9. The method claim 1, wherein each card includes an aperture in its middle for observing the behavioral response of the patient to the card.

10. A system for measuring a contrast sensitivity of a patient, the system comprising a plurality of cards, each card including a square wave grating having a spatial frequency and a contrast,
wherein the spatial frequency of each square wave grating is the same, and
wherein the contrast of each square wave grating is different.

11. The system of claim 10, wherein the spatial frequency is less than or equal to 0.250 cycles per degree.

12. The system of claim 10, wherein a size of each card is approximately 25.5 cm×56.0 cm.

13. The system of claim 10, wherein for each of the cards: the square wave grating occupies approximately one half of the card and a uniform gray color with a reflectance of 50% occupies approximately one half of the card.

14. The system of claim 10, wherein each square wave grating is tapered towards a center of its corresponding card.

15. The system of claim 10, wherein each card includes an aperture, and
wherein the aperture is operable to allow a person to see through the card.

16. An apparatus for measuring a contrast sensitivity of a patient, the apparatus comprising a substrate, the substrate including a low spatial frequency square-wave grating with a predetermined contrast.

17. The apparatus of claim 16, wherein the spatial frequency is less than or equal to 0.250 cycles per degree.

18. The apparatus of claim 16, wherein the square wave grating is tapered towards a center of the substrate.

19. The apparatus of claim 16, wherein the substrate includes an aperture, the aperture being operable to allow a person to see through the substrate.

20. The apparatus of claim 16, wherein the substrate is paper.

21. The apparatus of claim 16, wherein the substrate measures approximately 25.5 cm×56.0 cm.
